(19) United States
(12) Patent Application Publication
(10) Pub. No.: US 2024/0302365 A1
(43) Pub. Date: Sep. 12, 2024

(54) METHOD FOR DIAGNOSING CHRONIC URTICARIA

(71) Applicants: MD HEALTHCARE INC., Seoul (KR); AJOU UNIVERSITY INDUSTRY-ACADEMIC COOPERATION FOUNDATION, Suwon-Si, Gyeonggi-do (KR)

(72) Inventors: Hae-Sim PARK, Seoul (KR); Youngwoo CHOI, Suwon-si, Gyeonggi-do (KR); Yoon-Keun KIM, Paju-Si, Gyeonggi-do (KR)

(73) Assignees: MD HEALTHCARE INC., Seoul (KR); AJOU UNIVERSITY INDUSTRY-ACADEMIC COOPERATION FOUNDATION, Suwon-Si, Gyeonggi-do (KR)

(21) Appl. No.: 18/255,680

(22) PCT Filed: Nov. 12, 2021
(86) PCT No.: PCT/KR2021/016510

§ 371 Date: Jun. 2, 2023

(30) Foreign Application Priority Data
Dec. 4, 2020 (KR) 10-2020-0168285

(51) Int. Cl. G01N 33/564; G01N 33/68

(52) U.S. Cl. CPC G01N 33/564; G01N 33/6854; G01N 2333/212; G01N 2800/24

(57) ABSTRACT

Provided is a method for providing information on chronic urticaria and, more specifically, the provided is a method for diagnosing chronic urticaria by measuring an allergen-specific IgE antibody for a vesicle derived from Acinetobacter bacteria; a diagnostic composition using the same; and the like.

### TECHNICAL FIELD

The present invention relates to a method for diagnosing chronic urticaria, and more particularly, to a method for diagnosing chronic urticaria using an allergen-specific IgE antibody against Acinetobacter bacteria-derived vesicles; and the like.

### BACKGROUND ART

Allergy is a generic term for symptoms that are caused since the body's immune mechanism is in some cases more sensitive to foreign substances (antigens or allergens) although the foreign substances do not respond to most people when a subject is exposed to the foreign substances. Among them, an allergic skin disease refers to a disease that shows the skin symptoms caused by an allergic immune response, that is, a disease that shows symptoms such as pruritus, wheal, angioedema, erythema, and the like by allowing cells to generate antibodies against the foreign substances when the cells are exposed to the foreign substances, stimulating immune cells such as mast cells, macrophages, basophils, and the like, depending on the results of an antigen-antibody reaction, to facilitate the secretion of histamines, leukotrienes, prostaglandins, nitrogen monoxide (NO), and the like, and allowing these substances to induce an inflammatory response in the skin.

With the sudden global environmental change caused by the industrialization with the development of science, there is an increasing trend toward the frequency of allergic skin diseases such as chronic urticaria, atopic dermatitis, contact dermatitis, and the like. In recent years, it is believed that the allergic skin diseases account for 10% of the cases in Korea, including potential patients.

Among the allergic skin diseases, urticaria is generally a skin disease in which the skin swells up with a red or white color and is accompanied by severe pruritus since the plasma components of blood are temporarily accumulated in the tissue while increasing the permeability (a property of enabling the passage or invasion of material molecules) of the skin or mucosa's blood vessel. Accordingly, the urticaria may be classified into acute urticaria whose symptoms are improved within 6 weeks, and chronic urticaria whose symptoms last for 6 weeks or more, depending on the duration. This urticarial reaction may occur due to various diseases, and thus it is very important to accurately diagnose the chronic urticaria and use the corresponding therapeutic agent in order to treat the chronic urticaria. In general, a skin prick testing is performed to diagnose the allergy. In this case, the skin prick testing has limitations in that severe pain may be caused in patients during the diagnosis because it takes a long time to insert an injection needle through the skin and induce an allergic reaction, and it is difficult to make an accurate diagnosis because the results of a diagnosis may be different depending on the mode of injection by a diagnostician, and the like (Registered Korean Patent No. 10-1878414). Therefore, there is an increasing demand for a method capable of reducing the patients' pain and accurately and rapidly diagnosing the chronic urticaria.

### DISCLOSURE

### Technical Problem

The present invention is designed to solve the problems of the prior art, and therefore the object of the invention is to provide a method for providing information on chronic urticaria by measuring an amount of an allergen-specific IgE antibody against Acinetobacter bacteria-derived vesicles in a biological sample, a diagnostic composition using the same, a diagnostic kit, and the like.

However, a technical problem to be achieved by the present invention is not limited to the aforementioned problems, and the other problems that are not mentioned may be clearly understood by a person skilled in the art from the following description.

### Technical Solution

The present invention provides a method for providing information for the diagnosis of chronic urticaria, comprising: (a) measuring a level of an allergen-specific IgE antibody against Acinetobacter baumannii-derived vesicles in a biological sample; and (b) classifying a case in which the level of the measured allergen-specific IgE antibody increases compared to those in normal persons as the chronic urticaria.

According to one exemplary embodiment of the present invention, the biological sample may preferably be blood, plasma, serum, bone marrow, saliva, urine, feces, and the like, and more preferably blood, plasma, serum, and the like, but the biological samples are not limited as long as they include an IgE antibody.

According to another exemplary embodiment of the present invention, the measuring of the level of the allergen-specific IgE antibody may comprise Western blot, an enzyme-linked immunosorbent assay (ELISA), ImmunoCAP, a radioimmunoassay (RIA), radioimmunodiffusion, Ouchterlony immunodiffusion, rocket immunoelectrophoresis, immunohistostaining, an immunoprecipitation assay, a complement fixation assay, flow cytometry (a fluorescence activated cell sorter (FACS)), protein chips, and the like, but the methods are not limited as long as they can be used to measure an IgE antibody.

According to still another exemplary embodiment of the present invention, the subject classified as the chronic urticaria is characterized by using a steroid preparation as a therapeutic agent, and the steroid preparation may be an adrenocortical hormone, preferably a cortisol steroid, and more preferably a topical corticosteroid. In this case, as an analogue having a steroid structure, the steroid preparation is not limited as long as it may be used as a steroid therapeutic agent.

Also, the present invention provides a composition for diagnosing chronic urticaria comprising a substance for measuring a level of an allergen-specific IgE antibody against Acinetobacter baumannii-derived vesicles.

In addition, the present invention provides a kit for diagnosing chronic urticaria comprising a substance for measuring a level of an allergen-specific IgE antibody against Acinetobacter baumannii-derived vesicles.

According to one exemplary embodiment of the present invention, the substance may be preferably an antigen, an antibody, a peptide, an aptamer, or the like, which specifically binds to the allergen-specific IgE antibody against the Acinetobacter baumannii-derived vesicles. In this case, the substances are not limited as long as they may be a substance binding to the IgE antibody.

Further, the present invention provides a method for diagnosing chronic urticaria, comprising: (a) measuring a level of an allergen-specific IgE antibody against Acinetobacter baumannii-derived vesicles in a biological sample; and (b) classifying a case in which the level of the measured allergen-specific IgE antibody increases compared to those in normal persons as the chronic urticaria.

### Advantageous Effects

In a method for providing information on chronic urticaria according to the present invention, chronic urticaria can be diagnosed within a short time with high accuracy by measuring a concentration of specific IgE against Acinetobacter baumannii-derived vesicles in a biological sample, and the pain caused by the chronic urticaria can be minimized or reduced by applying a steroid as a therapeutic agent to patients diagnosed with chronic urticaria. Therefore, the method according to the present invention is expected to minimize the fast-growing medical costs and the pain caused by the chronic urticaria.

### DESCRIPTION OF DRAWINGS

FIG. 1 is a diagram showing the information on subjects registered in an experiment according to one embodiment of the present invention.

FIG. 2 is a diagram showing the results of measuring the total concentrations of IgE in normal persons and chronic urticaria patients according to one embodiment of the present invention.

FIG. 3 is a diagram showing the results of measuring concentrations of an allergen-specific IgE antibody against bacteria-derived vesicles in the normal persons and the chronic urticaria patients according to one embodiment of the present invention.

FIG. 4 is a diagram showing a ROC curve to check whether chronic urticaria may be diagnosed using the concentrations of the allergen-specific IgE antibody against the Acinetobacter baumannii-derived vesicles according to one embodiment of the present invention.

FIG. 5 is a diagram showing the results of determining an ability for the Acinetobacter baumannii-derived vesicles to cause an allergic reaction using a skin prick testing according to one embodiment of the present invention.

FIG. 6 is a diagram showing the results of determining an ability for the bacteria-derived vesicles to cause an allergic reaction using a skin prick testing according to one embodiment of the present invention.

FIG. 7 is a diagram showing the results of determining an effect of the Acinetobacter baumannii-derived vesicles on the early and late stages of chronic urticaria using the skin prick testing according to one embodiment of the present invention.

FIG. 8 is a diagram showing the results of identifying a therapeutic agent of chronic urticaria which is positive to the specific IgE against the Acinetobacter baumannii-derived vesicles according to one embodiment of the present invention.

### BEST MODE

In the method for providing information for the diagnosis of chronic urticaria according to the present invention, chronic urticaria may be diagnosed within a short time with high accuracy by measuring a level of an allergen-specific IgE antibody against Acinetobacter baumannii-derived vesicles, and the pain caused by the chronic urticaria may be minimized or reduced by applying a steroid as a therapeutic agent to patients diagnosed with chronic urticaria.

### MODE FOR INVENTION

In this specification, the term “biological sample” refers to all types of samples including IgE, which may be obtained in a non-invasive manner. Preferably, the biological sample may be blood, plasma, serum, bone marrow, tissue, cells, saliva, phlegm, urine, feces, and the like, and more preferably blood, serum, plasma, or the like, but the samples are not limited as long as they can be used to measure an amount of IgE.

In this specification, the term “vesicle (particularly, an extracellular vesicle)” refers to a structure composed of nano-sized membranes, which are secreted from various cells. In this case, the vesicle generally refers to all types of structures composed of membranes included in urine or blood, which are naturally secreted from cells or artificially produced in the present invention. Such vesicles may be used interchangeably with endoplasmic reticula, extracellular endoplasmic reticula, extracellular vesicles, exosomes, nanovesicles, micro-endoplasmic reticula, mircovesicles, and the like. The vesicles have a diameter of approximately 30 to 200 nm, and include various types of proteins, DNAs, mRNAs, micro-RNAs, and the like, which are derived from the cells in the vesicles. The vesicles may be isolated from urine or blood by using one or more methods selected from the group consisting of centrifugation, ultra-high speed centrifugation, high pressure treatment, extrusion, sonication, cell lysis, homogenization, freezing-thawing, electroporation, mechanical decomposition, chemical treatment, filtration by a filter, gel filtration chromatography, free-flow electrophoresis, and capillary electrophoresis. Further, a process such as washing for removing impurities and concentration of obtained vesicles may be further included.

In this specification, the expression “method for providing information” refers to a method for providing information on the diagnosis of chronic urticaria, that is, a method for obtaining information on the possibility to cause chronic urticaria when a level of specific IgE against the Acinetobacter baumannii-derived vesicles increases.

In this specification, the expression “measuring a level of an allergen-specific IgE antibody” refers to a process for determining whether an allergen-specific IgE antibody against the Acinetobacter baumannii-derived vesicles is present in a biological sample and determining a level of generation of the allergen-specific IgE antibody in order to predict the probability of being chronic urticaria. In this process, the presence of the allergen-specific IgE antibody against the Acinetobacter baumannii-derived vesicles may be determined by measuring an amount of IgE using an antigen, an antibody, a peptide, an aptamer, and the like, which specifically bind to the IgE. For this purpose, the presence of the allergen-specific IgE antibody against the Acinetobacter baumannii-derived vesicles may also be determined using a support to which the Acinetobacter baumannii-derived vesicles are fixed, or using a method for measuring IgE in the biological sample, which is bound to the support. An analysis method for this may include Western blotting, an enzyme-linked immunosorbent assay (ELISA), a radioimmunoassay, radioimmunodiffusion, Ouchterlony immunodiffusion, rocket immunoelectrophoresis, immunohistochemical staining, an immunoprecipitation assay, a complete fixation assay, flow cytometry (FACS), protein chips, an ImmunoCap assay, and the like, but the methods are not limited as long as they can be used to measure an amount of IgE.

In this specification, the term “kit” refers to an examination apparatus that may measure a level of an allergen-specific IgE antibody against Acinetobacter baumannii-derived vesicles in a biological sample to diagnose the onset of chronic urticaria. In this case, the kits are not limited as long as they may be used to measure an amount of the allergen-specific IgE antibody in the biological sample isolated from a patient. Preferably, the kit may comprise an antigen, an antibody, a peptide, an aptamer, and the like, which specifically bind to the allergen-specific IgE antibody. Also, the kit may further comprise a substance for measuring expression levels of constitutively expressed genes such as beta-actin, GAPDH, and the like for normalization.

In this specification, the term “subject (i.e., an individual)” refers to a target to which a steroid preparation may be administered. In this case, the targets are not limited as long as they are patients diagnosed with chronic urticaria by the method for providing information according to the present invention.

Hereinafter, preferred Examples for helping the understanding of the present invention will be suggested. However, the following Examples are provided only to more easily understand the present invention, and the contents of the present invention are not limited by the following Examples.

### EXAMPLES

### Example 1: Confirmation of Association of Acinetobacter baumannii-Derived Vesicles with IgE Secretion

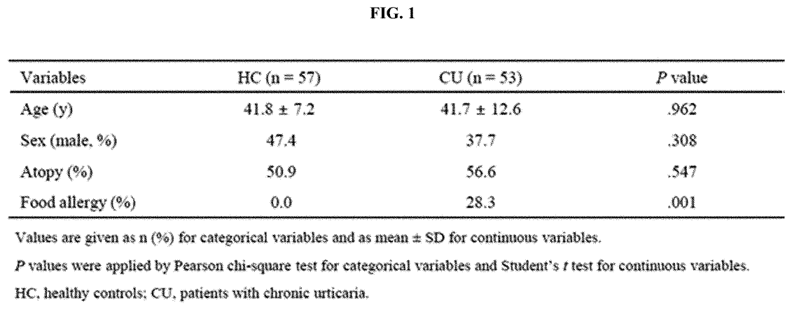

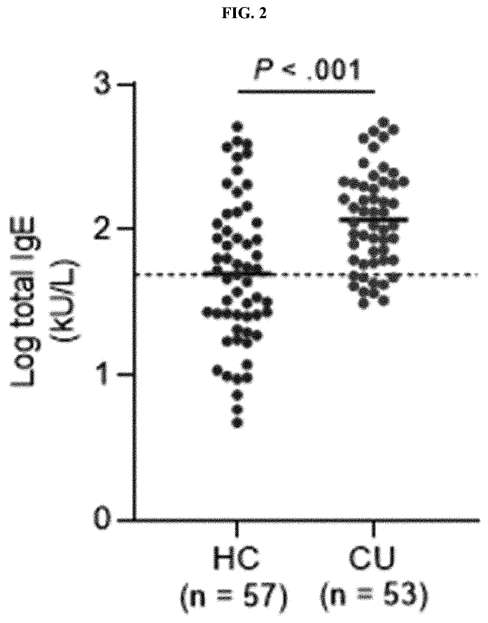

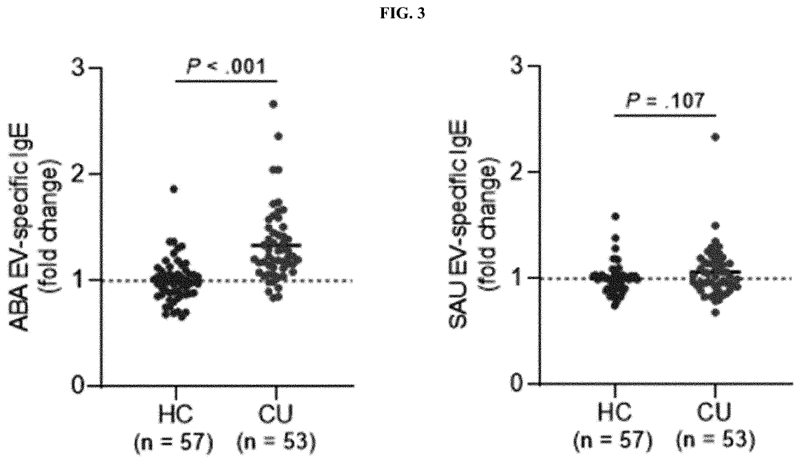

IgE is an immunoglobulin that plays an important role in the mast cell degranulation to secrete histamine in an allergic disease. To check an effect of Acinetobacter baumannii (ABA)-derived vesicles (particularly, extracellular vesicles) on the secretion of IgE, an experiment was performed for chronic urticaria (CU) patients and normal persons (healthy controls: HCs). More specifically, 53 CU patients and 57 HCs whose symptoms such as wheal, itching, angioedema, and the like lasted for 6 weeks or more were enrolled, and the information on the enrolled subjects is shown in FIG. 1. The total concentration of IgE in each of the patients' sera was measured using an ImmunoCAP system from ThermoFisher Scientifics. To measure an allergen-specific IgE antibody (i.e., specific IgE) against the bacteria-derived vesicles, a 96-well plate was then treated with 10 ng of each of Acinetobacter baumannii-derived vesicles and Staphylococcus aureus (SAU)-derived vesicles, reacted for 16 hours, and blocked using 1% bovine serum albumin. Thereafter, a concentration of IgE specific to the vesicles in the biological sample was measured using each of the fixed vesicles as an antigen and also using an anti-human IgE-peroxidase (Sigma-Aldrich). To obtain the bacteria-derived vesicles, a medium was inoculated with each of the bacteria, and cultured under the conditions of 37° C. and 200 rpm until the absorbance (OD_{600 nm}) reached 1.0 to 1.5. Thereafter, the culture solution was recovered, centrifuged at 10,000 g for 20 minutes to obtain a supernatant from which the cell bodies were removed. Then, the obtained supernatant was filtered through a 0.45 μm filter, and filtered again through a 0.22 μm bottle-top filter. The filtrate was then ultra-centrifuged at 150,000 g and 4° C. for 2 hours to obtain vesicular pellets. The obtained pellets were re-suspended in phosphate buffered saline (PBS) to obtain bacteria-derived vesicles. The obtained bacteria-derived vesicles were stored at −80° C. before use. Then, all the statistical analyses were performed using IBM SPSS software, and graphs were plotted using GraphPad Prism 8.0 software. The results are shown in FIGS. 2 and 3.

As shown in FIG. 2, it was confirmed that the total concentration of IgE significantly increased in the chronic urticaria patients (p<0.001).

As shown in FIG. 3, it was also confirmed that the concentration of the specific IgE against the Acinetobacter baumannii-derived vesicles significantly increased in the chronic urticaria patients (p<0.001), but that the concentration of the specific IgE against the Staphylococcus aureus-derived vesicles was not significantly different between the chronic urticaria patients and the normal persons. Staphylococcus aureus is a bacterium that is known to be associated with the cause of chronic urticaria, but it was confirmed that the concentration of the specific IgE was not significantly changed in Staphylococcus aureus.

Based on the results, it can be seen that the total concentration of IgE, particularly, the concentration of the specific IgE against the Acinetobacter baumannii-derived vesicles, increased in the chronic urticaria patients. Based on the results, it can be seen that the Acinetobacter baumannii-derived vesicles acted as an allergen in the chronic urticaria patients to increase the specific IgE against the Acinetobacter baumannii-derived vesicles. In particular, it can be seen that the secretion of IgE was further promoted compared to that of Staphylococcus aureus known to cause skin diseases.

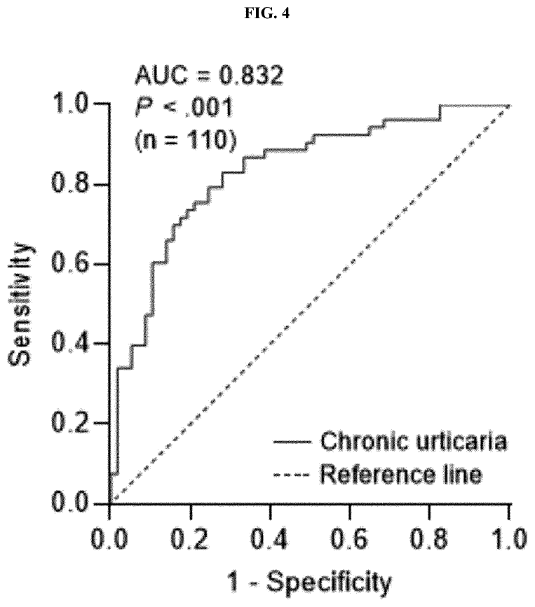

To check whether the chronic urticaria is diagnosed using the concentration of the specific IgE against the Acinetobacter baumannii-derived vesicles, a receiver-operating characteristic (ROC) analysis was performed. The results are shown in FIG. 4.

As shown in FIG. 4, it was confirmed that the specificity, the sensitivity, and the AUC were 71.9%, 83.0%, and 0.832, respectively, in diagnosing the chronic urticaria patients using the concentration of the specific IgE against the Acinetobacter baumannii-derived vesicles. Based on the results, it can be seen that the chronic urticaria might be diagnosed with high accuracy by measuring the specific IgE against the Acinetobacter baumannii-derived vesicles.

### Example 2: Confirmation of Relationship Between Acinetobacter baumannii-Derived Vesicles and Chronic Urticaria

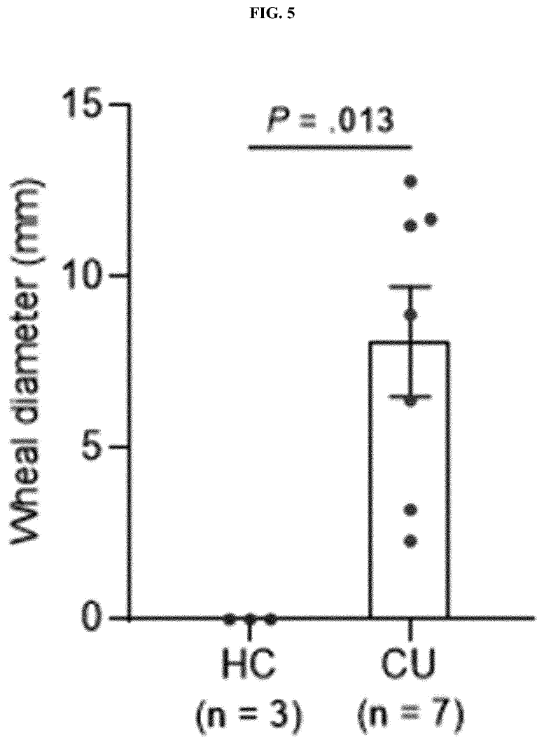

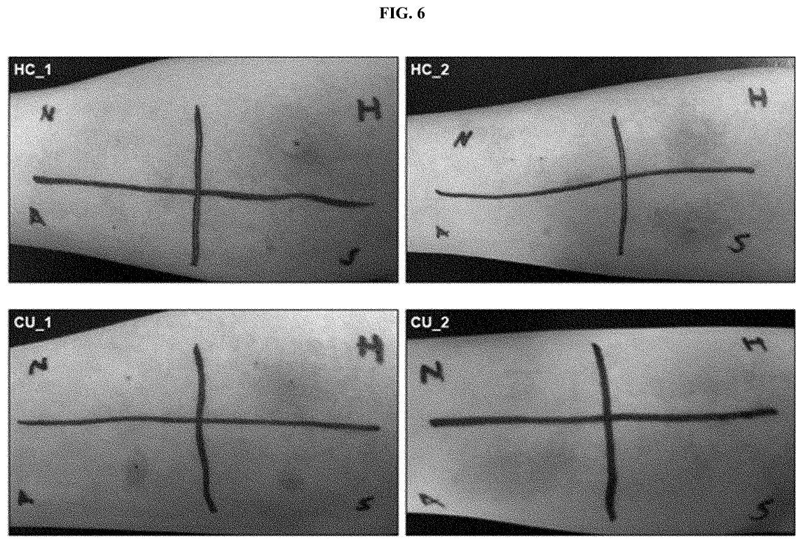

To confirm whether the Acinetobacter baumannii-derived vesicles cause chronic urticaria, a skin prick testing was performed. More specifically, a skin surface of the arm was stabbed with Acinetobacter baumannii-derived vesicles (A) or Staphylococcus aureus-derived vesicles (S) obtained in the same manner as in Example 1 at a concentration of 10 μg/mL. After 15 minutes, the diameters of wheals accompanied by erythema were determined. Saline and histamine were used as the negative (N) and positive (H) controls, respectively. The results are shown in FIGS. 5 and 6.

As shown in FIG. 5, it was confirmed that, when the Acinetobacter baumannii-derived vesicles were exposed to the skin, no wheals were observed in the normal persons, but the wheals accompanied by erythema were observed in all the chronic urticaria patients, and the diameters of the wheals were significantly increased compared to those in the normal persons. On the other hand, as shown in FIG. 6, it was confirmed that the wheals accompanied by erythema were partially observed or partially not observed in all the normal persons and the chronic urticaria patients in the case of the Staphylococcus aureus-derived vesicles, indicating that the Staphylococcus aureus-derived vesicles had no significant outcomes. Based on the results, it can be seen that the allergic reaction for the Acinetobacter baumannii-derived vesicles significantly increased in the chronic urticaria patients compared to the other allergens.

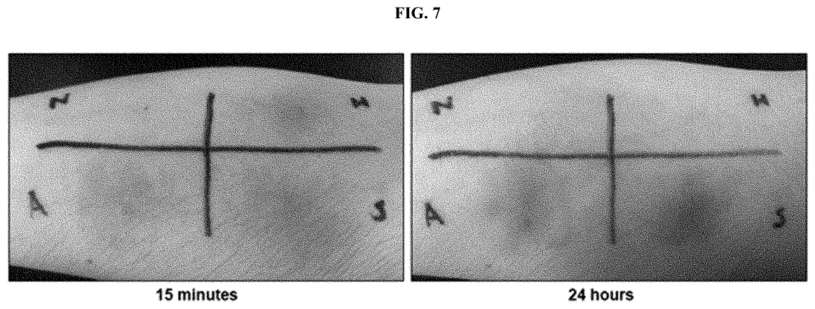

To check whether the Acinetobacter baumannii-derived vesicles has a long-term influence on the chronic urticaria, the skin prick testings were performed for the chronic urticaria patients, and then compared after 15 minutes and 24 hours. The results are shown in FIG. 7.

As shown in FIG. 7, it was confirmed that the wheals caused by histamine were observed at the beginning but disappeared after 24 hours, but the wheals accompanied by erythema increasingly lasted for 24 hours in the case of the Acinetobacter baumannii-derived vesicles. Based on the results, it can be seen that the Acinetobacter baumannii-derived vesicles were associated with the early stage of chronic urticaria as well as the late stage of chronic urticaria.

### Example 3: Identification of Therapeutic Agent of Chronic Urticaria Positive to Specific IgE Against Acinetobacter baumannii-Derived Vesicles

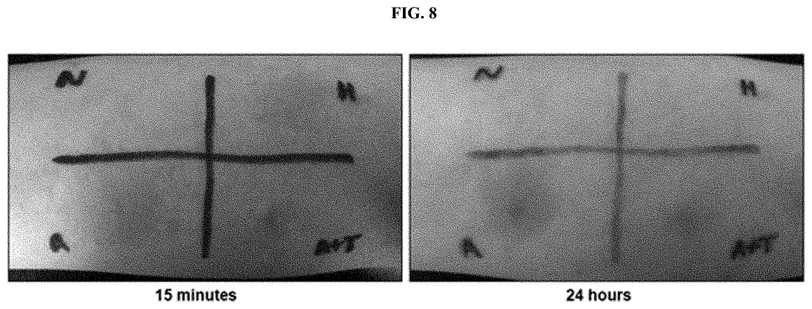

To identify a therapeutic agent of wheal accompanied by erythema caused by the Acinetobacter baumannii-derived vesicles, the skin was stabbed with the Acinetobacter baumannii-derived vesicles in the same manner as in Example 2, and then treated with a topical steroid (i.e., a topical corticosteroid). The results are shown in FIG. 8.

As shown in FIG. 8, it was confirmed that the wheals accompanied by erythema were effectively suppressed by the topical steroid. Based on the results, it can be seen that the steroid therapeutic agent was effective in the chronic urticaria patients diagnosed by measuring the specific IgE against the Acinetobacter baumannii-derived vesicles.

Based on the results, it can be seen that the chronic urticaria patients may be diagnosed within a short time with high accuracy by measuring the concentration of the specific IgE against the Acinetobacter baumannii-derived vesicles, and the pain caused by the chronic urticaria may be minimized or reduced by applying the steroid to the patients diagnosed with chronic urticaria.

The above-described description of the present invention is provided for illustrative purposes, and those of ordinary skill in the art to which the present invention pertains will understand that the present invention can be easily modified into other specific forms without changing the technical spirit or essential features of the present invention. Therefore, it should be understood that the above-described Examples are illustrative only in all aspects and are not restrictive.

### INDUSTRIAL APPLICABILITY

The method for providing information on chronic urticaria according to the present invention may be effectively used to diagnose and treat chronic urticaria patients because the chronic urticaria may be diagnosed within a short time with high accuracy by measuring the concentration of the specific IgE against the Acinetobacter baumannii-derived vesicles in the biological sample, and the steroid may be applied as the therapeutic agent to the patients diagnosed with chronic urticaria.

1. A method for diagnosing chronic urticaria, comprising:
(a) measuring a level of an allergen-specific IgE antibody against Acinetobacter baumannii-derived vesicles in a biological sample; and
(b) classifying a case in which the level of the measured allergen-specific IgE antibody increases compared to those in normal persons as the chronic urticaria.

2. The method of claim 1, wherein the biological sample is any one or more selected from the group consisting of blood, plasma, serum, bone marrow, saliva, urine, and feces.

3. The method of claim 1, wherein the measuring of the level of the allergen-specific IgE antibody comprises any one or more selected from the group consisting of Western blot, an enzyme-linked immunosorbent assay (ELISA), ImmunoCAP, a radioimmunoassay (RIA), radioimmunodiffusion, Ouchterlony immunodiffusion, rocket immunoelectrophoresis, immunohistostaining, an immunoprecipitation assay, a complement fixation assay, flow cytometry (a fluorescence activated cell sorter (FACS)), and protein chips.

4. The method of claim 1, wherein a steroid preparation is administered or applied as a therapeutic agent to the subject classified as the chronic urticaria.

5. A composition for diagnosing chronic urticaria comprising a substance for measuring a level of an allergen-specific IgE antibody against Acinetobacter baumannii-derived vesicles.

6. The composition of claim 5, wherein the substance is an antigen, an antibody, a peptide, or an aptamer, which specifically binds to the allergen-specific IgE antibody against the Acinetobacter baumannii-derived vesicles.

7. A kit for diagnosing chronic urticaria comprising a substance for measuring a level of an allergen-specific IgE antibody against Acinetobacter baumannii-derived vesicles.

8. The kit of claim 7, wherein the substance is an antigen, an antibody, a peptide, or an aptamer, which specifically binds to the allergen-specific IgE antibody against the Acinetobacter baumannii-derived vesicles.

9. (canceled)
